(19) United States
(12) Patent Application Publication
(10) Pub. No.: US 2022/0374916 A1
(43) Pub. Date: Nov. 24, 2022

(54) MANAGEMENT SOLUTIONS AND RELATED METHODS

(71) Applicant: 1 Source OHS, Inc., Gary, IN (US)

(72) Inventor: Frank Michael Messana, Hinsdale, IL (US)

(21) Appl. No.: 17/872,973

(22) Filed: Jul. 25, 2022

Related U.S. Application Data
(62) Division of application No. 15/582,298, filed on Apr. 28, 2017.
(60) Provisional application No. 62/329,053, filed on Apr. 28, 2016.

(51) Int. Cl. G06Q 30/00; G06Q 50/26

(52) U.S. Cl. CPC G06Q 30/018; G06Q 50/265

(57) ABSTRACT

A management system comprising a single master interface giving access to at least two modules selected from a list comprising a regulatory compliance module, an injury management module, a client relations management module, an employee health and safety module, a human relations module, and a project management module. A method of providing management services from the listed modules through a single system.

### CROSS-REFERENCE TO RELATED APPLICATIONS

This application is a Divisional of U.S. application Ser. No. 15/582,298, filed Apr. 28, 2017, and claims the benefit of U.S. Provisional Application No. 62/329,053, filed Apr. 28, 2016, both of which are incorporated herein by reference in their entirety.

### FIELD

This invention relates generally to systems, methods and apparatus relating to management, and, more particularly, to systems, methods and apparatus for managing occupational health, safety, and client relations needs for individuals and/or businesses and to assist them in improving their care and/or operations via better managed practices and related methods.

### BACKGROUND

Managing a company includes a wide range of activities including managing human resources, client relations, injuries, regulatory compliance, employee health and safety, as well as managing individual projects. Companies utilize a plurality of different systems to manage these different activities, each being provided through a different vendor. Employees of the company need to learn these plurality of different systems, need to deal with the plurality of vendors, and frequently need to do redundant work to keep each system up to date. With everything divided into so many systems individual tasks and deadline frequently fall through the cracks or get lost.

One major area of expense and use of management resources is handling occupational health and safety. Individuals need occupational health services in order to treat injuries that hamper their ability to work and/or reduce their overall ability to care for themselves. Some such injuries are workplace injuries. Currently, when an individual requires occupational medical services they go to a local clinic, and seek treatment or health services. For some larger companies, the human resources or safety department can help the employee find a proper clinic and fill out the appropriate paperwork. These human resources/safety departments may additionally be responsible for ensuring the employees and company are following appropriate regulations, keeping up to date with licensing and certifications, and utilizing safe practices.

Problems with the current system involve the lack of uniformity in process and in care and services as well as a lack of organization of the many different aspects of health and safety management. For a large company with locations all over the country and even all over the world, employees moving from location to location can be forced to learn a new system to get care or have to start over in seeking out care for themselves. Similarly, human resources/safety departments in these companies may lose track of licensing, certification, and regulation compliance requirements or deadlines as a result of the size and fluidity of their corporate structure. Additionally, two employees at different locations who suffer the same injury or need the same services can get disparate levels of care or services.

Accordingly, it has been determined that a need exists for comprehensive systems, methods and apparatus relating to management services including in the area of occupational health and safety services.

### BRIEF DESCRIPTION OF THE FIGURES

Embodiments of the invention are illustrated in the figures of the accompanying drawings in which:

FIGS. 1A-1B are a diagram illustrating the various exemplary modules of a management system according to embodiments of the present invention.

FIG. 2 is a flow diagram illustrating an exemplary injury management system according to an embodiment of the present invention.

FIGS. 3A-3B are a flow chart illustrating exemplary systems, methods, and apparatuses for use by a paramedic administering first aid according to an embodiment of the present invention.

FIGS. 4A-4B are swim lane flow charts illustrating exemplary systems, methods and apparatus for getting care using the occupational health and safety system discussed herein. FIG. 4A illustrates exemplary steps that may be taken or required leading up to the appointment (e.g., Pre Visit). FIG. 4B illustrates an exemplary process of using the occupational health services, methods or apparatus after the appointment has been completed (e.g., Post Visit).

FIGS. 5A-5B illustrate alternative exemplary occupational health services, methods and apparatus in accordance with some embodiments of the invention. FIG. 5A illustrates exemplary steps that may be taken or required leading up to the appointment (Pre Visit). FIG. 5B illustrates an exemplary process of using the occupational health services systems, methods and apparatus after the appointment has been completed (Post Visit).

FIGS. 6A-6B illustrates exemplary occupational health and safety systems, methods and apparatus in accordance with some embodiments of the invention. FIG. 6A illustrates the steps leading up to the appointment (Pre Visit) and FIG. 6B illustrates the process of using the occupational health services after the appointment has been completed (Post Visit).

FIG. 7 is a flow diagram illustrating an exemplary regulatory compliance system according to an embodiment of the present invention.

FIGS. 8A-8E illustrate screenshots from an exemplary customer portal created in accordance with some embodiments of the invention and showing the steps of creating a new order.

FIG. 9 illustrates a system layer diagram of occupational health/safety services, methods and apparatus in accordance with some embodiments of the invention.

FIG. 10 illustrates a cycle diagram for exemplary systems, methods and apparatus in accordance with some embodiments of the invention.

FIG. 11 is a flow diagram illustrating an exemplary clinic management system according to an embodiment of the present invention.

FIG. 12 is a flow diagram illustrating a method administering care.

FIG. 13 is a block diagram of a of a health and safety management system for phone triage injury first aid screening, management, and reporting according to an embodiment present invention.

Elements in the figures are illustrated for simplicity and clarity and have not necessarily been drawn to scale or to include all features, options or attachments. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

Similar features shown in the different embodiments illustrated in the figures above share similar reference numbers. Each element has a three digit reference number, with the first digit representing the embodiment number and the last two digits representing the component. Other than the differences explicitly described and/or shown, elements with corresponding elements are understood to be substantially similar.

### DESCRIPTION OF THE INVENTION

Many variations of management systems and related methods are discussed herein and even further are contemplated in view of this disclosure. The management systems discussed herein are designed to provide a single system with a single master interface through which one or more users can access a plurality of modules each designed to facilitate one aspect of management. For example, a single system includes modules for managing one or more of regulatory compliance, injury management, client relations management, employee health and safety, and project management. The single master interface provides a single source to access documents, communications, calendars, electronic training, contact lists, and several other resources from each of these aspects. By all being in one system, it also allows information to be automatically shared in areas of overlap and allows management to ensure that all record keeping (e.g., email and other electronic record data storage, invoicing, etc. will be handled in one approved way or format). In some applications, the internal management, such as a human resources and/or safety department employee, uses the master interface to access all of the modules and manage those areas. Alternatively, the system can be used by a third party to handle several managerial tasks, such as creating regulatory compliance programs, managing client relations, managing human resources tasks, organizing injury management, etc. This third party can be an administrator working for the provider of the system, or can be a separate entity entirely. When managed by an outside third party, the consolidation of several business services to one master interface further streamlines the processes so that the third party manager has an easier time managing these services and can more easily take on more responsibilities creating economies of scale for management and the third party manager. Below, occupational health and safety applications are used for exemplary purposes, but it should be understood that the management system and related methods described are not limited to occupational health and safety applications and can be used in all aspects of management or management services.

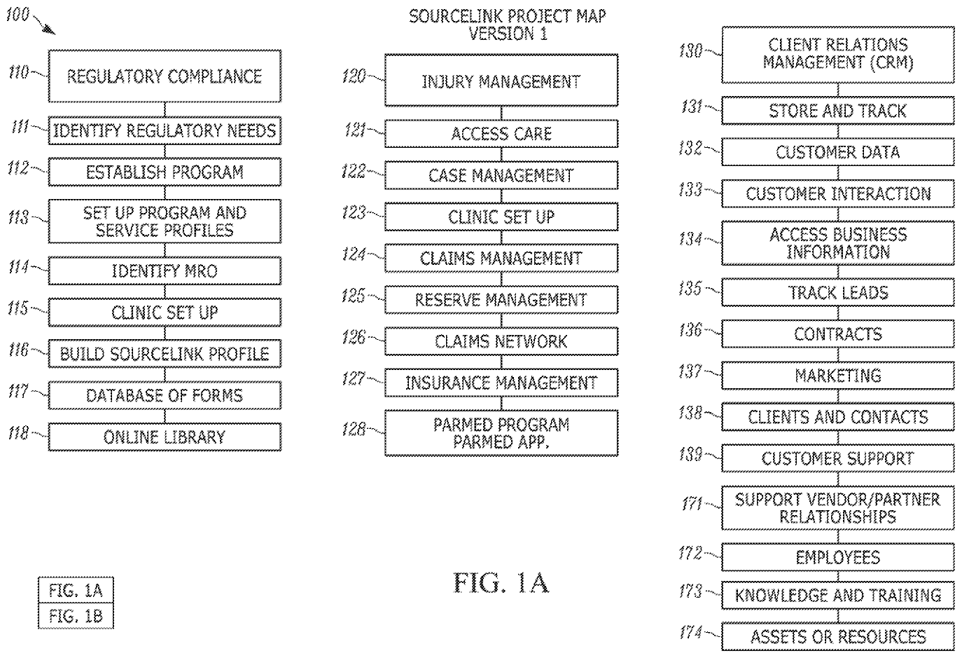

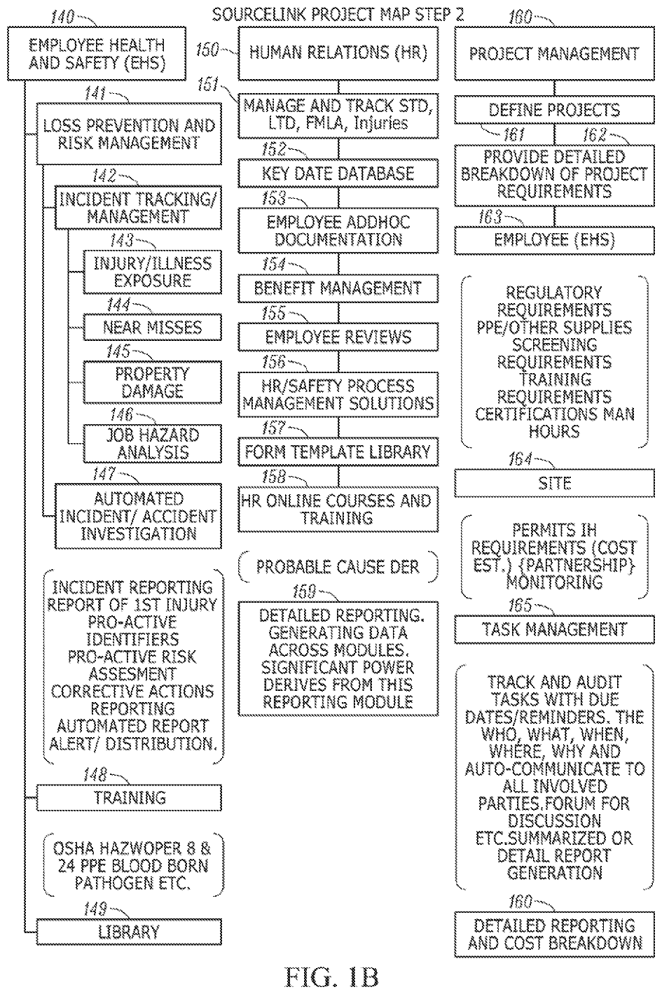

FIGS. 1A-1B illustrate exemplary modules, all or some of which are managed by a single system. The first exemplary module is a regulatory compliance module 110. The first step of regulatory compliance 110 is to identify regulatory needs 111. Various industries can be regulated by a wide variety of entities. Government (at federal, state, and/or local levels), parent companies, industry groups, international bodies, ratings organizations, and consumers are all potential sources of regulation. To identify regulatory needs 111, the management system 100 compiles all of the regulatory requirements, or at least all of the regulations related to occupational health and safety, from each source into one location.

Once all of the regulatory requirements are identified, the system 100 establishes a program to meet said requirements in step 112. The program varies based on the regulatory needs identified in step 111. Additionally, the program varies from employee to employee. Some regulations are site specific, activity specific, or based on what materials the employee is likely to encounter in the course of their job. The established program identifies which requirements are relevant to each individual employee and tailors a custom plan accordingly. The plans include tasks to be completed at certain points in employment, such as pre-employment, random testing, testing as a result of probably cause, post-accident/incident testing, and follow up testing.

In step 113, the system 100 sets up program and service profiles. The program and service profiles are profiles that can be viewed through a user terminal, such as a computer, with access to the system. A profile includes an overview of the program or service, including a basic overview of when it is applicable and what it entails. For example, a profile might identify the program as being for machine operators and including periodic drug and alcohol tests.

In step 114, the system 100 identifies a medical review officer (MRO). An MRO is a medical professional that reviews and analyzes tests, such as drug and alcohol tests, to interpret the results. Having an MRO pre-identified by the system 100 streamlines the administration of tests by removing the necessity of human resources to identify and contract with MROs on their own. If an existing MRO becomes unavailable, such as by retiring, the system 100 can automatically identify a replacement from a network of known MROs in the area.

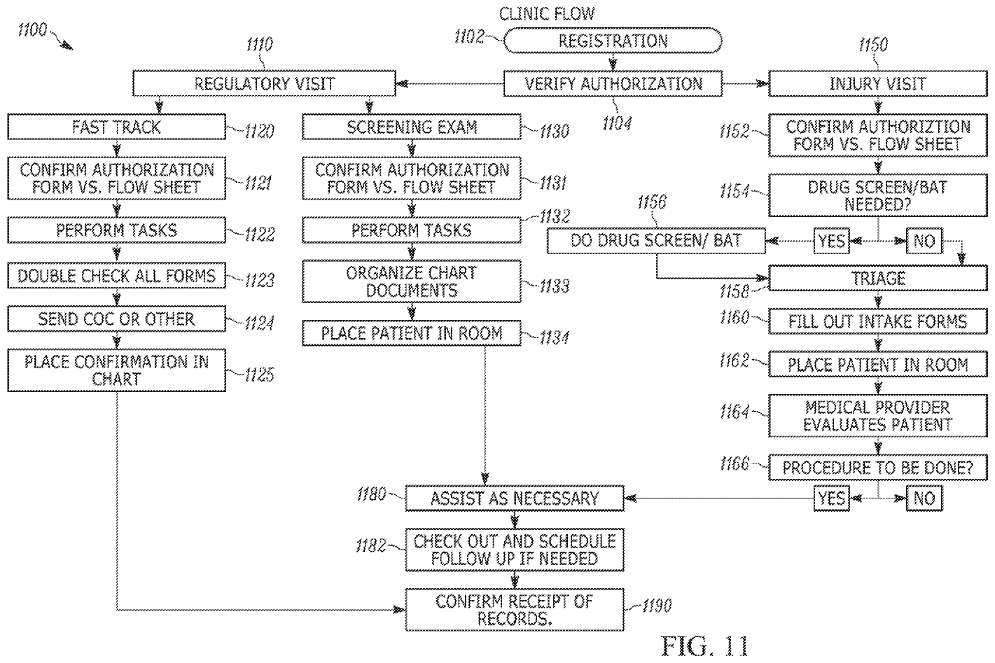

Similarly, the system 100 sets up a clinic in step 115. The system 100 has a network of clinics from which it identifies one best suited for the needs of the company based on location, price, and capability. If no suitable clinic is already in the network, clinics can be added as described in FIG. 11 and corresponding description below.

In step 116, the system 100 builds a profile in the software, such as SourceLink™, for each of the users (the employer, employee, clinic, and MRO). The software facilitates communication between the users by way of their profiles. The profiles also include information about each user that is visible to other users or system administrators based on access. For example, an employee profile may include some medical history information that is only visible to their clinic or MRO or to system administrative staff. Depending on what type of medical information is included in the profile, the system encrypts the data to comply with relevant regulations, such as HIPAA.

The system 100 additionally compiles a database of forms 117 and an online library 118 to provide easy access to users. The online forms are easily accessed by users through their user terminals and are all located in one place so they are easy to find. The forms may also be linked or transmitted with appointments or other events. For example, when the system 100 is used to schedule a drug test, the relevant forms are automatically provided to the employer, employee, and/or clinic so that they can be filled out and submitted within the system 100.

In one example, regulation requires employees to be tested for drugs and alcohol. The system 100 identifies the specific requirements from each relevant regulation source and forms a program that meets all of them. The program then identifies an appropriate clinic and MRO to carry out the drug and alcohol tests for the company. Finally, the system automatically schedules each required test, provides the appropriate forms, generates invoices and/or bills and transmits them to the correct parties.

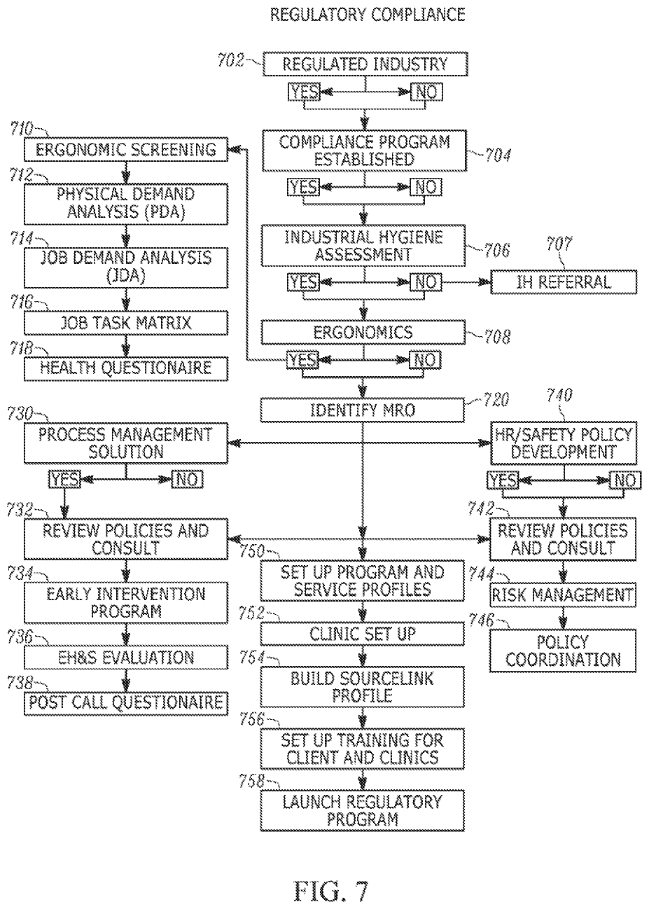

FIG. 7 illustrates a flow diagram or decision tree 700 illustrating exemplary operation of the regulatory compliance module 110. First it is determined if the industry is a regulator industry 702. As described above, an industry may be regulated by government of any level, partners, parent companies, industry groups, consumers, certifying organizations, or other entities. Next, it is determined if a compliance program has already been established 704. One type of compliance program is an industrial hygiene assessment program. An industrial hygiene assessment program identifies potential environmental factors in the workplace that may cause injury or illness and determines ways to prevent or control the risk. If no industrial hygiene assessment plan is in place 706, the system 100 refers the user to an IH specialist 707. As with clinics and other vendors, the system includes a network of IH specialists. If no suitable IH specialist is already in the network, additional IH specialists can be added.

Some workplaces require certain ergonomic regulation 708. If ergonomic regulation is required, the system walks the user through performing ergonomic screening 710, physical demand analysis 712, job demand analysis 714, and job task matrices 716. In some forms, some of these steps are automated by the system based on industry and/or occupational standards. Finally, the system 100 provides health questionnaires 718 to the employees.

The regulatory compliance system additionally identifies an MRO 720 to oversee the plan, such as by reviewing medical tests and questionnaires. As discussed herein, the system includes a network of MROs which can be referred based on a number of factors. If no network MRO is suitable, an additional MRO can be added to the network.

Regulatory compliance further includes developing process management solutions policies 730, HR/Safety policies 740, and setting up a compliance program 750. If the client already has process management solutions policies in place, an administrator reviews the existing policies and consults to improve the policies 732. This may include implementing an early interventions program 734 and evaluating the EHS policies 736. After the consultation, the user is provided with a post call questionnaire 738 to determine if the consultation was useful in improving the policies.

Similarly, if the client already has HR/Safety policies, an administrator reviews the policies and consults 742. This includes ensuring there is a risk management system 744 and helping coordinate the policies 746.

Finally, the system 100 combines all of the regulatory needs to create a master plan. This includes setting up a relationship between the client and a clinic 752 to perform required tests and treatments. A profile for the client and the plan are then built 754 and added to the system 100 so that it can be accessed through user terminals. Once the profiles are in place, electronic training through the system is set up for both the client and the clinic to aid both in carrying out the plan to comply with the regulations 756. Finally, the program is launched 758.

In some forms, the system 100 additionally includes an injury management module 120. The injury management module 120 facilitates procedure in cases of an injury. Injury management 120 includes two main objectives, access care 121 and case management 122. The first step in the case of an injury is to provide access to care to the injured party. Access care 121 involves first determining the seriousness of the injury through phone triage and/or first aid. The system provides a series of questions and tests to either a medical professional performing phone triage or a paramedic performing first aid in order to help identify the injury. The tests are limited to standard diagnostic tests, such as the Glasgow Coma Scale, and/or standard first aid practices. The system 100 does not provide actual medical treatment. If it is determined that treatment is needed, the system 100 refers the injured party to a clinic or instructs the user to dial 9-1-1 for emergency treatment if necessary.

The case management step 122 tracks and organizes treatment of the injury from start to finish. When an injury occurs, it is entered into the system as a new event. It can be entered by the injured party themselves, the paramedic that administered first aid, or a supervisor or human resources employee. Any activity taken to treat the injury, such as the first aid administered, is entered into the system under the case file.

The system 100 is a single source that manages making appointments with clinics 123, insurance claims 124, reserve management 125, claims networks 126, and insurance management 126. In operation, when an injury necessitates treatment through a clinic, the system 100 suggests one or more clinics from the network as described above. If no clinics meet the requirements, or if the user requests a different clinic, a new clinic can be added to the network as described below. The system 100 also manages insurance 126 to ensure that the insurance policy is sufficient and up to date. When a user visits a clinic, the system 100 receives a bill from the clinic and makes an insurance claim 124. The system 100 additionally bills the employer and/or employee for their portions of the bill (such as a copay or deductible).

For self-insured or partially self-insured companies, the injury management module 120 managed the amount of money held in reserve as anticipated or potential expenses arise from injuries. Active management of the reserve fund ensures compliance with regulation as well as quickly freeing up funds when an injury requiring reserve is treated.

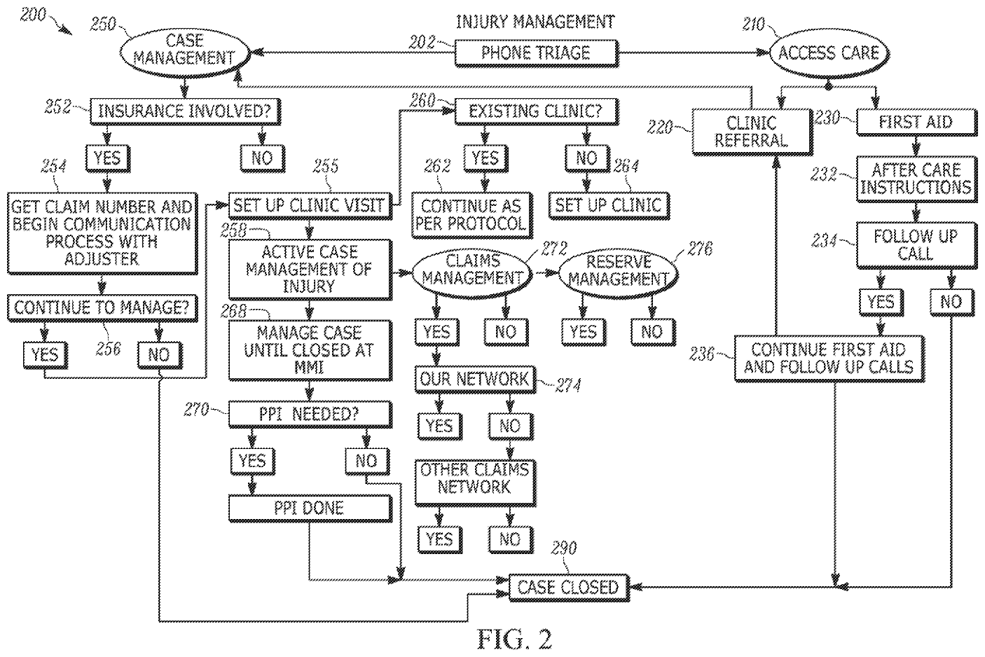

FIG. 2 is a flow diagram or decision tree illustrating the injury management use of an exemplary injury management 200. If an injury is determined to not be serious enough to necessitate seeking emergency help, then it should first be managed by phone triage 202. Either the injured employee or another employee calls a number relating to the system 100 which automatically connects them to a medical professional (such as a nurse, physician, or physician's assistant) from the network. In a preferred form, the medical professional has a terminal to access the system 100. The medical professional asks a series of questions to gauge the seriousness of the injury so that they can report the event. The questions are targeted towards information gathering, not administering medical aid. The phone triage establishes the injured parties name, the employer, the site, the project, the activity, the manager, the shift, the job title, etc. to ensure that the event gets propagated to the appropriate modules. The medical professional also gets a description of the injury itself as well as how the injury occurs. The medical professional gauges the state of the employee to determine if additional injury management is needed. This includes asking the employee the level of pain they are in, whether they feel they can continue working (either full duty or light duty or fully duty with specific restrictions). It further includes inquiring about preexisting conditions that may be aggravated by the injury. The medical professional can further schedule a follow up call to check on the status of the employee at a later time to see if the injury quickly healed or persisted.

If the medical professional or the employee determine that additional injury management is needed then they use the system 100 to both seek access to additional care 250 as well as to manage the case 210.

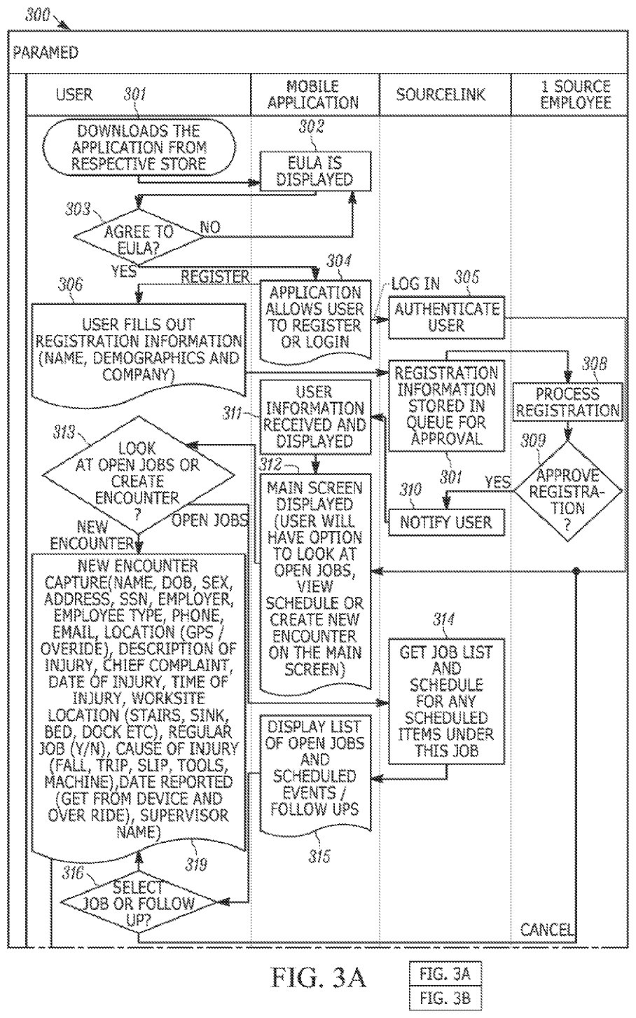

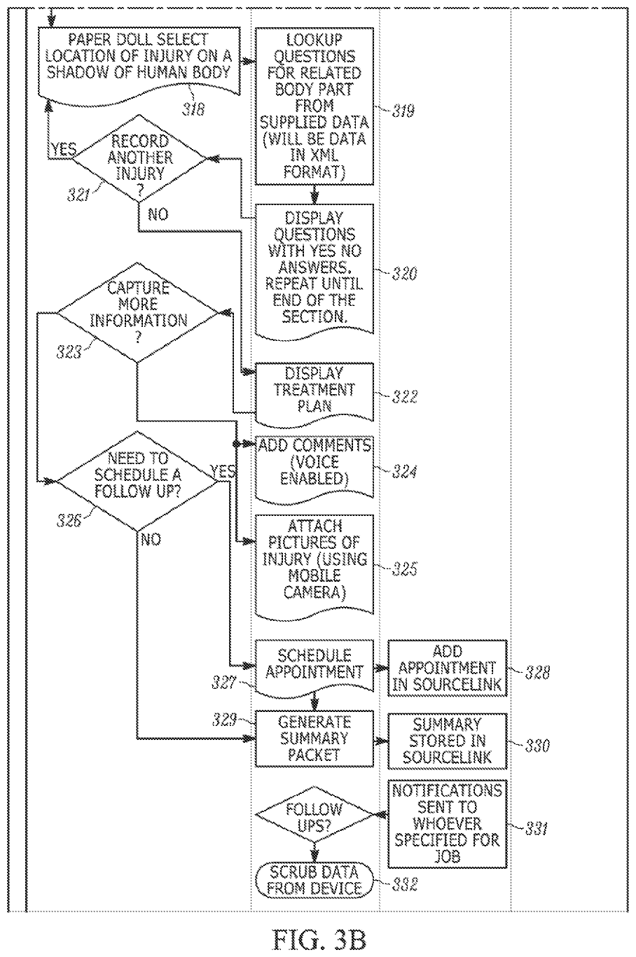

Access to care 210 includes access to immediate, on-site care, such as first aid 230, and/or arranging offsite care via clinic referral 220. If first aid 230 is needed a paramedic or other employee with first aid training is contacted and sent to the injured employee. In one form, the paramedic has access to a portable user terminal having a paramed app to help walk them through the first aid. An exemplary paramed app is described in greater detail in FIG. 3 and related discussion below. The paramedic does not provide full medical care, only first aid. The first aid involves administering basic standard tests to try to diagnose the injury, such as the Glasgow Coma Scale to test for brain injury, or testing motor control and sensitivity of various body parts to test for nerve damage. The first aid 230 optionally includes administering of basic treatments, such as over the counter medication, bandaging, ice and/or cold packs, stretching, etc.

After first aid 230 the paramedic provides after care instructions 232 to the injured party. This may include continuing the basic treatment discussed above, switching to light duty, resting, etc. If necessary, a follow up call 234 is scheduled so that the paramedic can check on the healing progress. If it is determined that no follow up call 234 is necessary, or after the follow up call or calls and continued first aid are completed to the point no additional care is needed, the case is closed 290.

Alternatively or additionally to the first aid 230, the system 100 can be used to refer the injured employee to a clinic 220. As discussed above, the system 100 includes a network of clinics that can be recommended based on a number of factors, such as price, location, and capabilities of treating the specific injury. If no network clinics are suitable, clinics from outside of the system can be referred instead. Network clinics have a terminal to access the system, and as such the system automatically sends the clinic the information collected during phone triage as well as other relevant information from the employee profile. The system 100 additionally aids in scheduling the appointment and sends reminders to the employee when the appointment nears.

Case management 250 involves managing the logistics of injury management, such as handling insurance claims and scheduling treatment. The first step of case management 250 is determining if insurance covers the injury and/or if it is necessary to file an insurance claim 252. If insurance is used the system contacts the insurer to acquire a claim number and communicates with the adjuster 154. It next must be determined if the injury is fully managed, or if continued management is required 156. Once the injury is fully managed, the case is closed 290.

If continued management is needed, the system 100 sets up a clinic visit 258 as described above. If an existing network clinic is suitable to manage the injury 260 it is immediately scheduled 262. If no network clinic is suitable, a new clinic is added to the network 264 as described below.

Cases where active management is necessary 266, the case continues to be managed until the maximum medical improvement (MMI) is achieved 268. Once MMI is achieved, it is determined if the employee is fully healed or if they have a partial permanent impairment 270.

If there is partial permanent impairment (PPI), a PPI process is entered to compensate the employee and equip them for living with the PPI. If they are fully healed, the case is closed 290.

Active management also includes may include actively managing the claims 272. The first step of managing the claims is determining if the claims are all in network 274. In network claims can be invoiced automatically by the system 100. Out of network claims for clinics can also be managed automatically with the system 100. Laws regarding insurance claim amounts vary from state to state. Some states provide a fee schedule, which is stored in the system and used in automatic invoicing. Other states rely on “usual and customary” costs which can also be managed by the system.

Returning to FIG. 1A, a client relations management module 130 provides a number of tools to help a user maintain a positive relationship with clients/customers. The system 100 stores and tracks customer activities 131 as well as compiles customer data 132 including business information 134. This information is used to better understand the needs of the user so that they can be more effectively met. The system 100 also provides a means for customer interaction 133. In addition to the automated messaging, such as reminders. The system 100 has private messaging options to securely send messages to other users, such as between users and their clients. For example, when the user is working on a long project for or with a customer, the customer can be provided a customer portal to the system from which they can access the project management module. Through this module they are kept up to date on progress, deadline, cost projections, etc. if authorized by the user.

The system additionally serves as a single source to access a number of tools to expand to new clients. These tools include lead tracking 135, contracting 136, and marketing 137. These tools help track the timeline and expenses associated with lead tracking and marketing, and help develop programs to do so more efficiently. This helps ensure that target markets are marketed to with the proper saturation of advertising to optimize return. Having lead tracking and marketing managed by the same module can detect trends among potential clients and help tailor the marketing to target them.

As described above, the system 100 includes tools for communication which can be used by users to communicate with their clients as well as with vendors. These include clients contact lists 138, customer support portals 139, and vendor/partner relationship tools 171. By sending all communications through the system 100, it provides a single log so that ensure that no communication falls through the cracks. For example, communications between an administrator and a client on a specific event will be stored under that event so that they can be easily accessed by the client or administrator at any time. The customer support portals 139 include FAQs, direct messaging, as well as real time messaging (text based and/or voice).

The client relations management module also includes tools to help the user learn to fully utilize the system 100 and specifically the client relations management modules 130. They include user portals for employees 172, knowledge and training tools 173, and other assets and resources 174. Through these tools the user, and employees thereof can access electronic training on marketing and client relations, access information on the clients or potential clients, view previous communications, contracts, or projects worked on for said client, etc.

Turning to FIG. 1B, the employee health and safety (EHS) module 140 provides features than can prevent the number of incidents and accidents and therefore reduce the cost of injury management. These tools include loss prevention and risk management tools 141 such as incident tracking/management 142, injury/illness exposure 143, lists of near misses 144, lists of property damage 145, and job hazard analysis 146. The loss prevention and risk management tools 141 are all designed to track trends in order to identify risks and/or causes of loss. For example, a list of near misses 144 all while the employee was undertaking a certain activity or using a certain piece of equipment can identify said activity or equipment as a hazard before any real loss or injury is caused. As a result, steps can be taken to implement a safer method or add safety features to the machine to prevent future injuries. Similarly, tracking illness exposure 143 can be used to identify pockets of illnesses which can then be used to determine the source. For example, a spike in illness among employees that work in a certain location within the site may indicate an environmental cause (such as mold or chemicals).

EHS 140 additionally automates incident/accident investigation 147. Whenever an incident is reported into the system 100, the EHS module identifies the appropriate employee or department to investigate said incident and creates a program for them to do so. This includes sending reminders, providing relevant information, and generating forms to be completed by the investigator. Until the forms are completed and submitted, the event remains open and the department as well as human resources and/or management are periodically reminded. In some forms, the automatic investigation 147 identifies trends as discussed above and when a trend is identified it prioritizes the investigation.

Training tools 148 are provided to train employees in safe practices. These include OSHA training programs, training on how to use hazardous materials, training on the proper use of dangerous equipment, training on avoiding blood borne pathogens, etc.

The EHS module 140 includes an electronic library 149 in which all health and safety related documentation can be accessed. This includes spec sheets for chemicals, manuals for equipment, documents describing safe practices, etc. Access to this library 149 enable employees to self-educate, such as by looking up how to safely use new equipment. It also provides a single source so that materials can be quickly located when needed, such as quickly looking up spec sheet of a chemical when it is spilled.

In addition to aiding human resources deal with regulatory compliance 110, injury management 120, and EHS issues 130, the system 100 can include a human resources module 150 to aid in the other aspects of HR. This includes providing a single system to do much of the organizing and bookkeeping involved in HR, such as managing and tracking short and long term disability, family and medical leave, and injuries 151, compiling a database of key dates 152, compiling a database of employee AddHoc documentation 153, managing employee benefits 154, and keeping employee reviews 155. A human resources associate can simply access the module 150 through a user terminal and select an employee. Under the employee profile there will be key dates 152 such as deadlines for outstanding paperwork, dates for tests and certifications, employment anniversaries, schedule vacations, etc. It will also display the number of sick days or disability days available 151 and any employee documentation (e.g., contracts, benefits forms, etc.). Through the benefit management tools 154, the human resources associate can check and change an employee's benefit options, such as changing their retirement plan contribution or health care plan enrolment. Employee reviews 155 are created and/or stored on the system so that they can likewise be accessed by navigating to the employee's profile. In one form, the system 100 automatically sends out employee review forms to managers for each of their employees at the appropriate time, and then automatically compiles the results to be either sent or read to the employee. These compiled results are then stored in the employee profile for future reference.

HR/safety process management solutions 156 are tools to help implement safety processes. This includes new safety procedures and incident/accident reporting and investigation as discussed above. Similarly to previous modules, the HR modules also provides a library of forms 157, access to electronic courses and training 158, and automated reporting 159. Providing a single source in which all of these forms 157 and course 158 can be accessed drastically decreases time and overhead. HR associates can quickly find whatever form or course they are looking for by merely using the user terminal to access the system 100 and navigating to the proper module. A search feature within these libraries makes finding the appropriate form or course even faster. In some forms, the courses are automatically sent to the HR associate and/or other employees based on a calendar. For example, an annual ethics course may be sent out to each employee required to complete it on a certain date with periodic reminders until it is completed.

The automated reporting of data across modules 159 reduces time and overhead further. With existing practice in which each of the modules discussed herein would be one or more independent systems, the system 100 contains each module. Therefore, when data is entered into a single module, it is automatically propagated throughout the other modules. For example, if an employee is injured and the event is entered through the injury management module 120, the details of the injury are automatically passed onto the EHS module and the HR module. This way the information only needs to be entered once, and all of the modules remain up to date and accurate.

Many of the modules discusses above are maintained on a companywide or site wide basis. However, much of this information may also be relevant when narrowed to a specific project. The system 100 further includes a project management module 160 for organizing and aiding in said projects. Similar to regulatory compliance 110, the project management module 160 can be used to define 161 any type of project and then provide a breakdown of the requirements 162 to complete said project. For example, if a large project is entered with a specific due date, the module 160 generates milestones and stores those milestones in the calendars of the associated employees. The module can additionally break down the overall project into individual steps that can be checked off as completed.

The project management module 160 includes its own EHS submodule 163. The EHS submodule operates in much the same way as the EHS module 140. The system 100 will propagate entered into either the EHS module 140 or the EHS submodule 163 to the other as discussed above. For example, when reporting an injury or near miss through the EHS module 140, the user indicates that it was in the course of working on the project and the EHS submodule 163 for that project is updated.

The project management 160 aids in a number of aspects of project management including site management 164, task management 165, and reporting 166. Site management includes providing and storing permits for the site, tracking deadlines to submit said permits, monitoring IH requirements, estimating costs to lease or maintain the property or costs to work with a partner on the site, and monitoring the site's use, such as entry logs. Task management 165 is similar to the project management module 160 as a whole only for managing individual tasks within the project. The task management tools 165 define the task and the deadlines associate therewith, and automatically track due dates in order to send reminders to the relevant employees. The task management tools 165 also help facilitate communication between employees working on the task, such as by generating a forum. Automatic reports 166 on the progress, costs, estimated time of completion, and other aspects of the project are automatically generated and distributed by the system 100. This aids in keeping all parties well informed on the progress in order to improve oversight. By automatically generating reports, areas in which the project is falling behind deadlines or exceeding its budget can be quickly detected such that adjustments can be made to correct in a timely fashion.

As discussed above, the system 100 provides a single portal to a plurality of the exemplary modules described. By having the plurality of modules on a single system, the time and overhead required is reduced by eliminating redundant work. All information entered into the system through any module is automatically spread to each other module as needed. Additionally, the system 100 provides a centralized location where users receive reminders, reports, and communications.

In some embodiment, the system 100 described above further includes a paramedic portal. The paramedic portal is an application for use on a portable computing device, such as a tablet or smart phone, by paramedics. When a paramedic is called onto a site of a customer business in response to an injury they can access the application. The application displays guidelines describing how to provide first aid for specific jobsite injuries, that were developed by experts as discussed above. The application can further display the records of the injured participant, including if they are currently in any treatment and if so how to provide first aid in light of their current status in the treatment process. The guidelines further assist the paramedic in determining whether or not the participant requires further emergency medical attention. If further emergency medical attention is required, the paramedics can transport the participant to a hospital. One benefit of this embodiment is to prevent unnecessary transports of participants. Many insurance providers base their premiums on the number of workplace injuries that require a specific level of treatment. In the United States, these are known as OSHA recordable injuries. The need to transport a patient after hours can significantly increase the risk of triggering an OSHA recordable incident. Thus, by using the system disclosed herein a business is able to reduce the number of unwarranted OSHA recordable incidents and prevent its premiums from going up due to same.

In addition to providing guidelines, the paramedic portal displays forms generated by the system that may be required for the situation at hand. For example, if the person receiving first aid indicates they do not want to be treated or transported to a hospital or clinic for further assessment, the portal may display a declined treatment/transport form that the paramedic can have the person receiving first aid sign to confirm these were their instructions for this episode. Other forms may be provided for the paramedic to sign, such as if they determined that transport and/or further assessment/treatment either was or was not necessary. The forms can be signed on a touch screen, and then the signed forms will be saved in the customer file on the system so that it can be easily found at a later date if requested by an insurance agency or regulatory agency (such as OSHA). Thus, one advantage of the systems, methods and apparatus disclosed herein is the ability to reduce the number of OSHA recordable events in order to help the business keep its premiums down and, for example, utilize that savings to provide better benefits to its employees, thereby yielding a better managed system offering better services.

FIGS. 3A-3B are a flow chart illustrating an exemplary application for administering first aid under the injury management module as described above. First, the app is downloaded by a user onto their mobile device 301. The first time the app is opened, an end user license agreement is displayed 302 that must be agreed to by the user 303 before the user can continue. Once agree to, the user either registers or logs in 304. If the user logs in with an existing account, the user is authenticated 305 and then the homepage is displayed 312.

If the user registers a new account, they must first fill out registration information 306 (including name, position, employer, etc.). The system 100 stores the registration information and forwards it to an administrator 307. An administrator processes the registration information 308 and approves the new user 309. The system 100 notifies the user when they have been approved 310. The application then displays the new user information 311. The user can then access the main page 312. From the main page, the user can either look at open jobs or create a new encounter 313. Creating a new encounter requires the user to acquire and then input information 317 about the injured party as well as the injury, similar to the information collected via phone triage above. If instead the user selects to view an existing job, the system 100 loads scheduled items for the job 314 and displays the information via the app 315. The user can then select a different job or select to follow up on the displayed job 316.

Once a job is selected, or encounter created, a paper doll of a human body is displayed by the app. The user selects a location on the body corresponding to the injury sustained by the employee 318. Based on the selection, the app loads a series of questions 319 to help diagnose the injury. In a preferred form, the database of questions are stored on the portable device such that they can be loaded without internet access. The app displays the questions one at a time 320. In some forms, the questions displayed are based on previous answers.

The app is not designed to aid in medical treatment, only in first aid. The questions are standard questions or tests to make basic diagnoses and provide first aid. For example, the displayed questions walk the user through determining the Glasgow Coma Scale and based on the answers determine if there is brain damage. Alternatively, the questions walk the user through testing the motor control and sensitivity of an injured body part to check for nerve damage.

Once all of the questions are answered, the user can select to return to the paper doll to select an additional injury 321 in which case it returns to step 318 above and repeats until all injuries have been input.

Once there are no more injuries to select, the app displays a treatment plan 322. As discussed above, a treatment plan only consists of basic first aid, such as over the counter medication, stretching, light duty, applying cold/heat, etc. Alternatively, the treatment plan may be to seeking either immediate emergency care or scheduling a clinic visit.

After sharing the plan with the injured party, the user can capture additional information 323. This includes adding comments 234, either text comments or voice comments, and/or taking pictures 325.

Finally, the user determines if a follow up appointment is needed 326. If an appointment is needed, the app displays a calendar to so that an appointment can be scheduled 327. The scheduled appointment is uploaded to the system 100 which automatically propagates it to the employees profile as well as the users profile so that they will both be reminded 328.

The application then generates a summary packet 329 of the encounter. The summary packet is uploaded to the system 100 and stored in memory 330. The system 100 automatically notifies the proper employees (such as a manager) 331. If there were no follow ups scheduled, the app deletes the data on the encounter from the portable device 332.

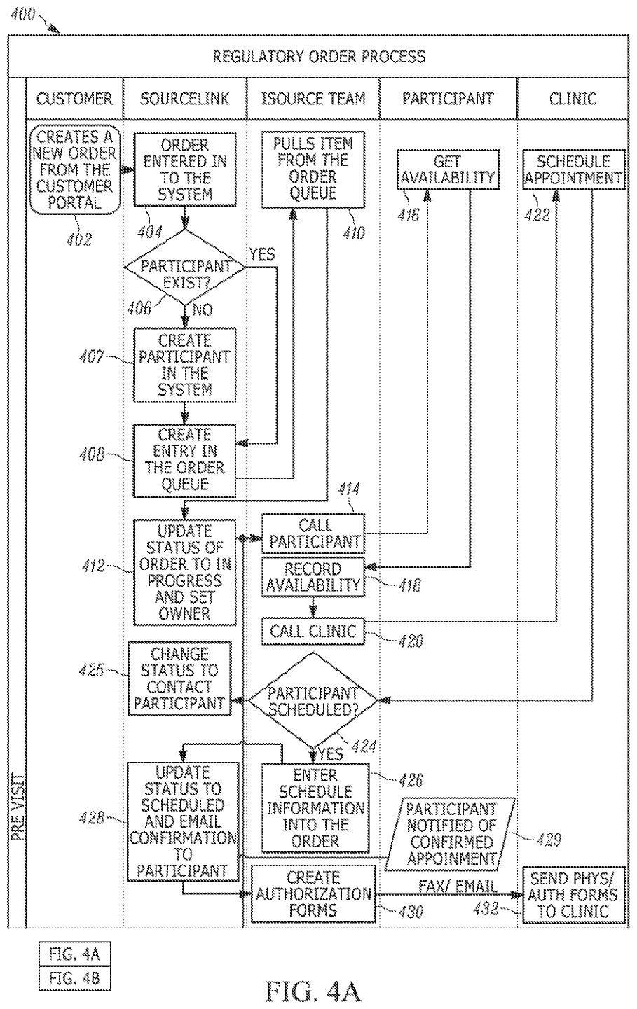

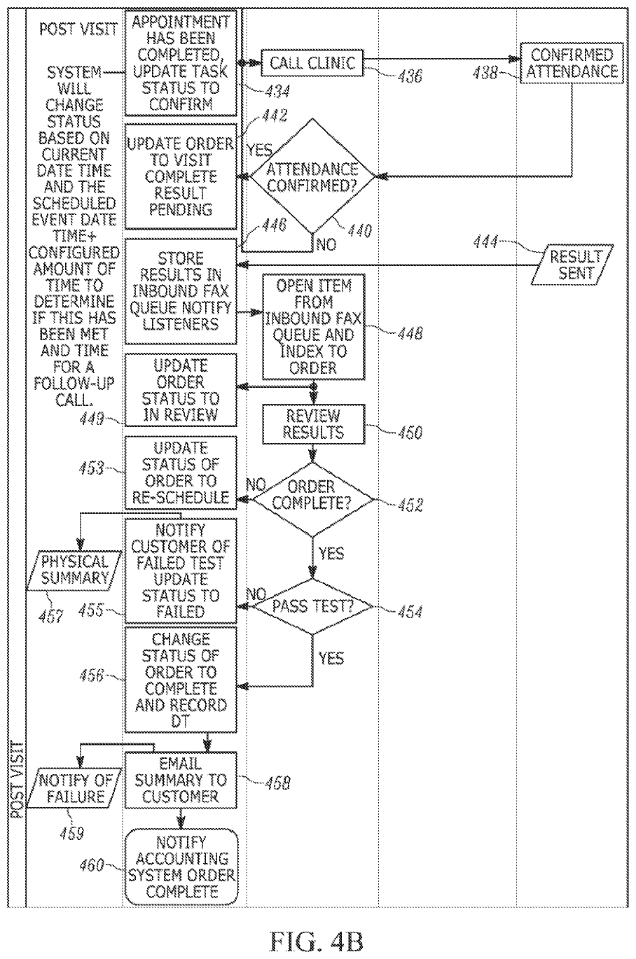

FIGS. 4-6 are example methods of occupational therapy systems and methods relating to same under the injury management module described above. FIGS. 4A-4B are flow charts illustrating a method 400 of getting care using the occupational therapy system discussed herein. FIG. 4A illustrates the steps leading up to the appointment. In step 402, an end user creates a new order on a customer portal. A customer portal is a computing device (e.g., fixed or mobile), such as a personal computer, tablet, or smartphone in communication with the system servers. The customer can refer to either the employee seeking care (the participant) or a representative of the company that creates the requests on behalf of injured employees, such as a human resources representative.

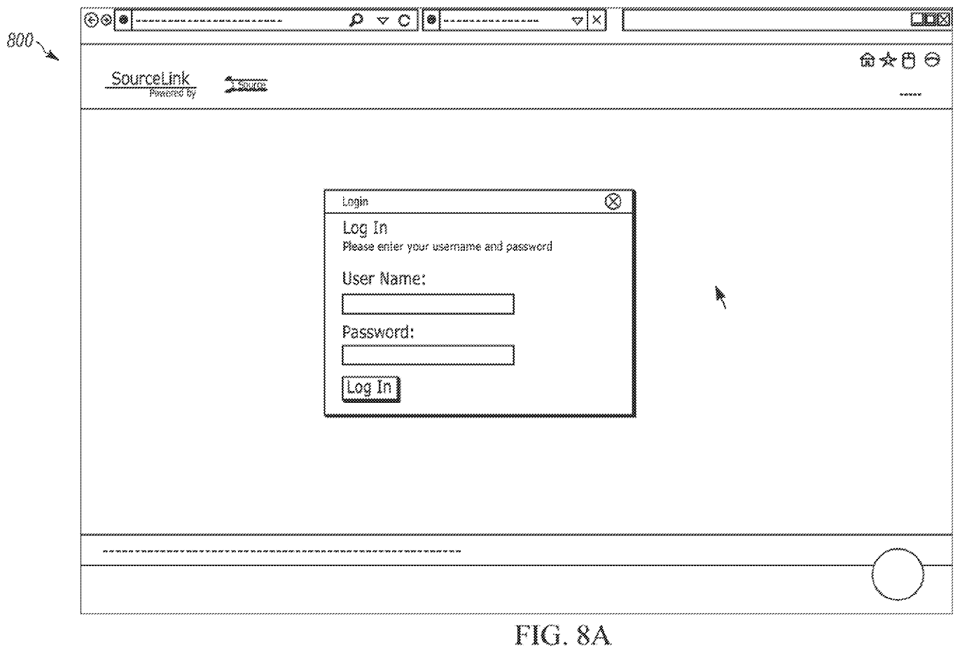

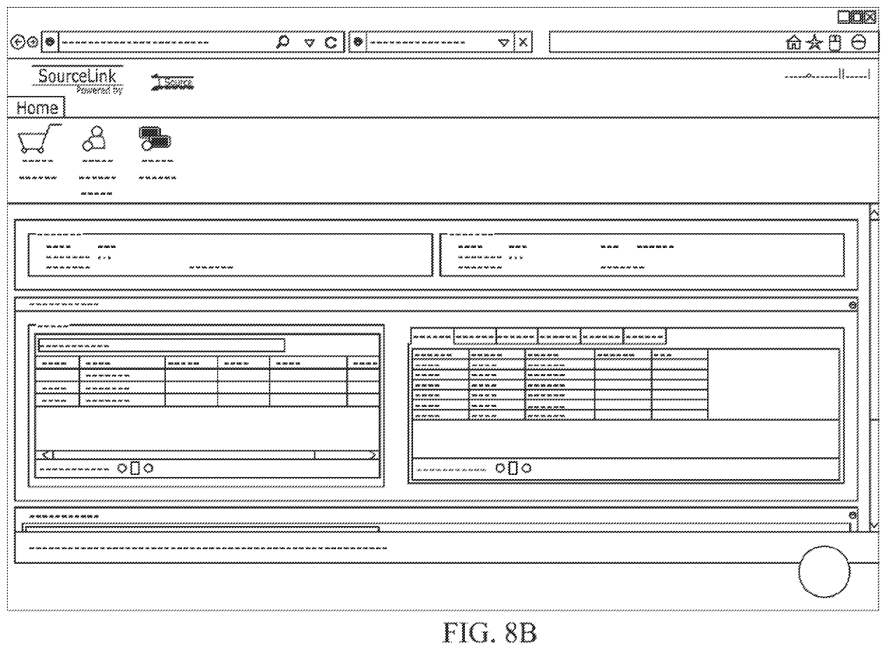

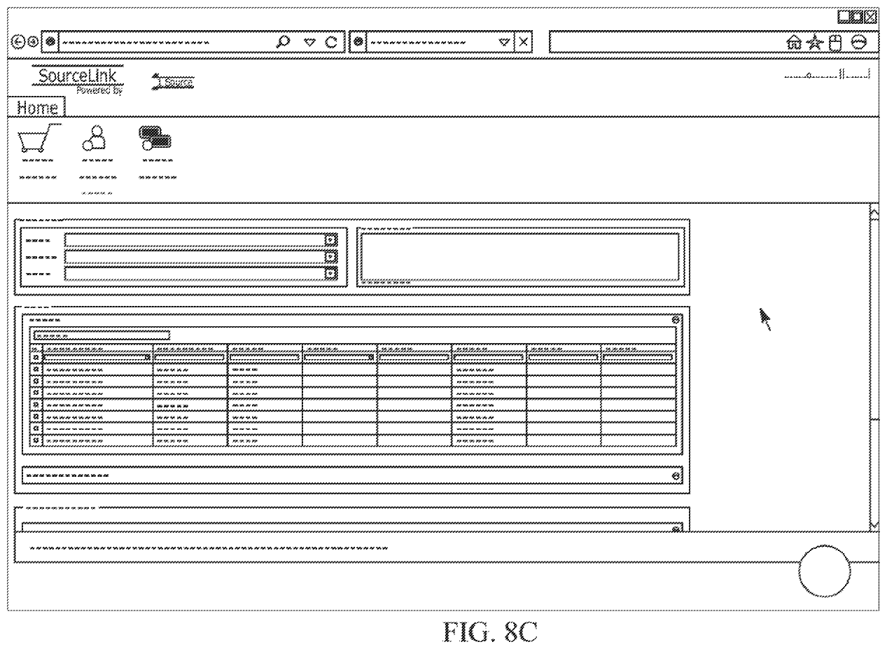

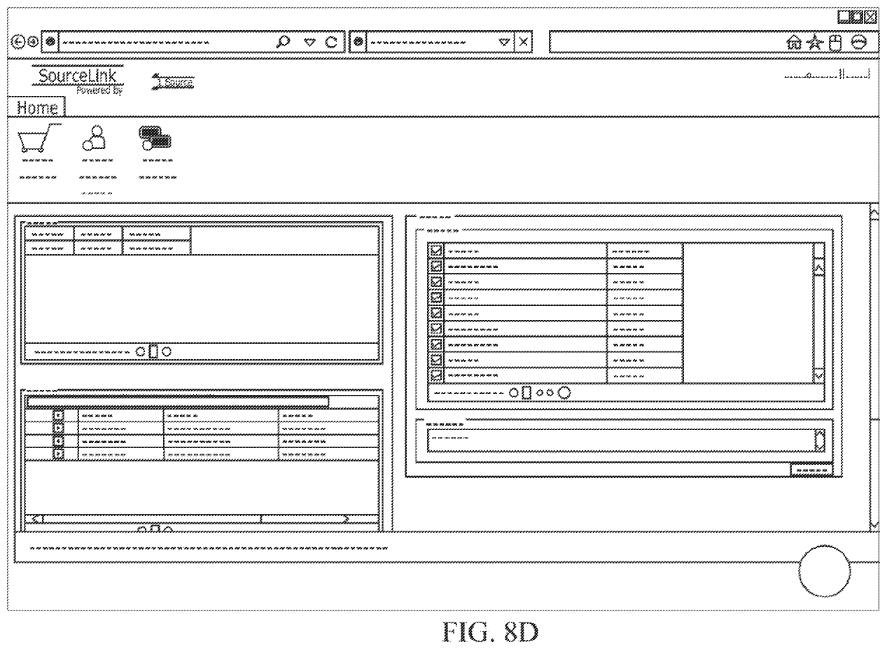

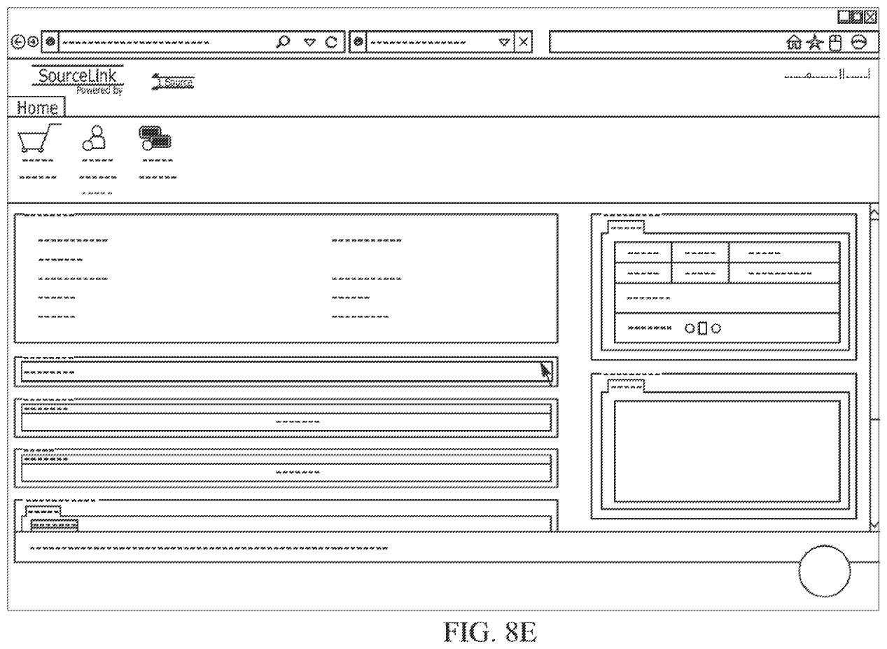

FIGS. 8A-8E illustrate example screenshots from the customer portal showing the steps of creating a new order. First, turning to FIG. 8A, the customer logs in using the company profile. Turning to FIG. 8B, the company profile will be associated with a list of known locations and employees of the company stored on the system servers. This list is generated during setup of the system and can be edited at will to remain current or accurate. From this page, the customer can also see service profiles, orders, and invoices for each location or for each employee. FIG. 8C shows the actual new order creation screen. From this screen, the customer can select the location, order type, and profile for the batch of orders. They can then select the employees to create orders for and select the individual procedures to be completed on each order. Once submitted this batch contains 1 order for each employee selected. After the order is created, the customer has a chance to review the order before submitting it as shown in FIG. 8D. The customer can select the batch, then select an order in the batch, review all the selected information and correct any of the orders. Once complete they submit the order (step 404 in FIG. 4A). After the order is received in the system, a confirmation page is displayed on the customer portal as shown in FIG. 8E. On the confirmation page, all of the information about the order can be seen and notes can be added to the order. Also, any correspondence about that order will be tracked here. The customer can see the procedure details of the order, any upcoming or historical visit information about the order, any invoices generated for the order and the overall completion of the order.

Turning back to FIG. 4A, the order is received at the system in step 404. In the present figures, the occupation health system is referred to as “SourceLink™”. The system determines if the participant or participants listed in the order are already listed in the system as employees of the customer, step 406. If they are not, then a new profile is automatically generated for the new participant in step 407. If the participants are in the system, or after they are added, the system places the order in a queue of pending orders in step 408.

In the present embodiment, an employee or team member of the company managing the occupational health system pulls orders from the queue and reviews them reviews them in step 410. In the present embodiment, these employees are referred to as the “1 Source Team™”. In alternative embodiments, some or all of the steps taken by the team member can be automated. Once a team member has pulled the order form the queue, the system updates the order status, as displayed on the customer portal, to in progress in step 412. In step 414, the team member contacts the participant directly in order to proceed with the order. The participant provides the team member with their availability to seek treatment in step 416. Availability can include both times they are available, as well as locations they are capable of reaching, and any extenuating circumstances (such as preexisting conditions or religious beliefs) that can affect what treatments and treatment providers can be used.

In step 418, the team member records the availability information provided by the participant, and with this information calls a clinic in step 420. The clinic is selected from a network of clinics stored in the system. Clinics are included in the network based on location, hours of operation, quality of care, and the type of care given. Clinics include occupational therapy clinics, as well as solo practitioners. It can also include other types of medical care providers, such as hospitals, outpatient care centers, physical rehabilitation centers, and chiropractors. Before being added to the network, clinics can be audited by a team member. The auditing includes evaluating the services offered, the facilities, the equipment, the hours of operation, and the staffing. If no clinic currently in the system is suitable to fulfill the new order, new clinics can be added to the network in response to the order by undergoing the auditing process described above.

Every clinic in the network is provided with guidelines on how to treat various injuries. The guidelines are developed based on the opinions of medical professionals working for or on behalf of the company operating the occupational health system and/or from medical organization such as the American Medical Association, State medical boards, etc. The guidelines can differ from national medical guidelines by either being stricter or by preferring one appropriate treatment over others. The guidelines serve to provide uniform care to all participants across every location in which the customer operates. However, the clinic will have the freedom to stray from the guidelines on a case by case basis when they feel it is appropriate based on their medical expertise and/or is allowed under jurisdictional practice regulators (e.g., State Boards, FDA, etc.).

In step 422, the clinic attempts to schedule an appointment based on the ability of the participant. The team member determines if an appointment was made in step 424. If not, they change the status of the order to “contact participant” in step 425, and then return to step 414 to confirm availability and discuss other options. If the appointment is scheduled, the team member enters the schedule information into the system in step 426. They then update the order status in step 428 and notify the participant in step 429. The participant can be notified by any communication method, such as email and telephone.

After notifying the participant, the team member creates authorization forms in step 430 and sends them to the clinic in step 432. At the scheduled time, the participant goes to the clinic in order to receive treatment.

FIG. 4B illustrates the process of using the occupational therapy system after the appointment has been completed. Once the scheduled time of the appointment passes, the system automatically updates the status to indicate that the appointment is complete in step 434. A team member then calls the clinic in step 436 so that the clinic can confirm the attendance of the participant (step 438). The team member receives this confirmation in step 440. If the participant did not attend, the team member returns to step 414 (see FIG. 4A) and contacts the participant to check availability to schedule a new appointment. If the participant did attend, the team member updates the status on the system to “visit complete” in step 442.

At some point after the visit, the clinic sends the results of the visit to the system in step 444. Upon receipt, the system automatically saves the results under the appropriate order so that they can be accessed by team members, the customer, and/or the participant in step 446. The system further notifies listeners that results have been received. Listeners can include team members assigned to the order, the customer, and/or the participant. A team member then takes the results in step 448 and updates the status of the order to indicate that the results are under review in step 449. In step 450, the team member reviews the results. In step 452, the team member determines from the results if the order is complete. If not, the status is updated in step 453, and the process returns to step 408 so that the next step of the order can be scheduled. If the order is complete, the team member determines if the participant passed any tests required in step 454. Example tests can include physicals used to determine if the participant is ready to return to work. If the test is failed, the order status is updated in step 455 and the customer is notified in step 457. If the test is passed, the order status is changed to “complete” in step 456. The system then automatically emails a summary to the customer in step 458, which is received by the customer in step 459. The system finally notifies the accounting system that the order is complete 460.

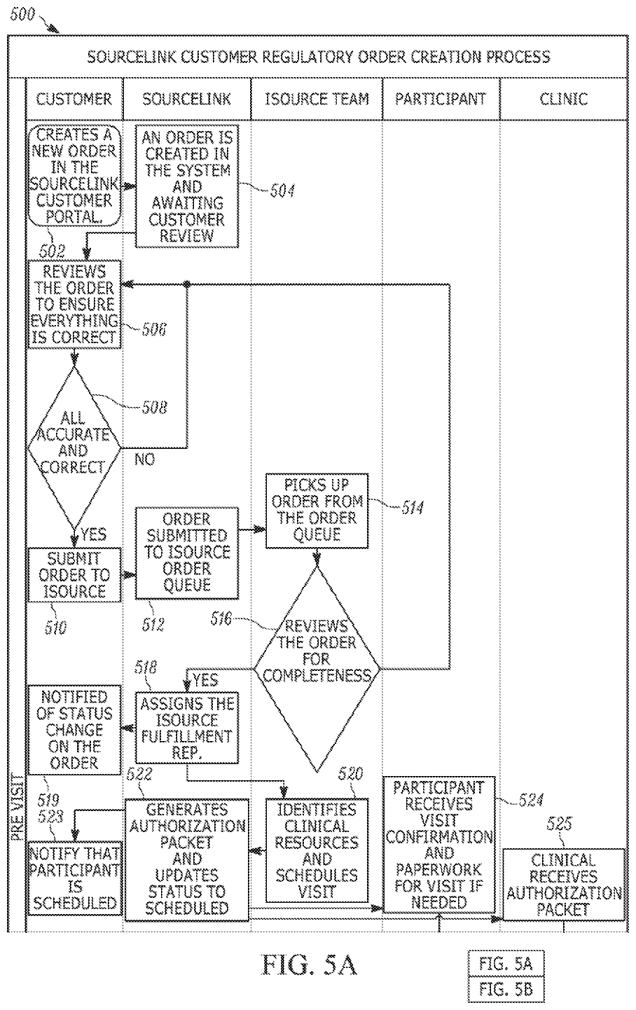

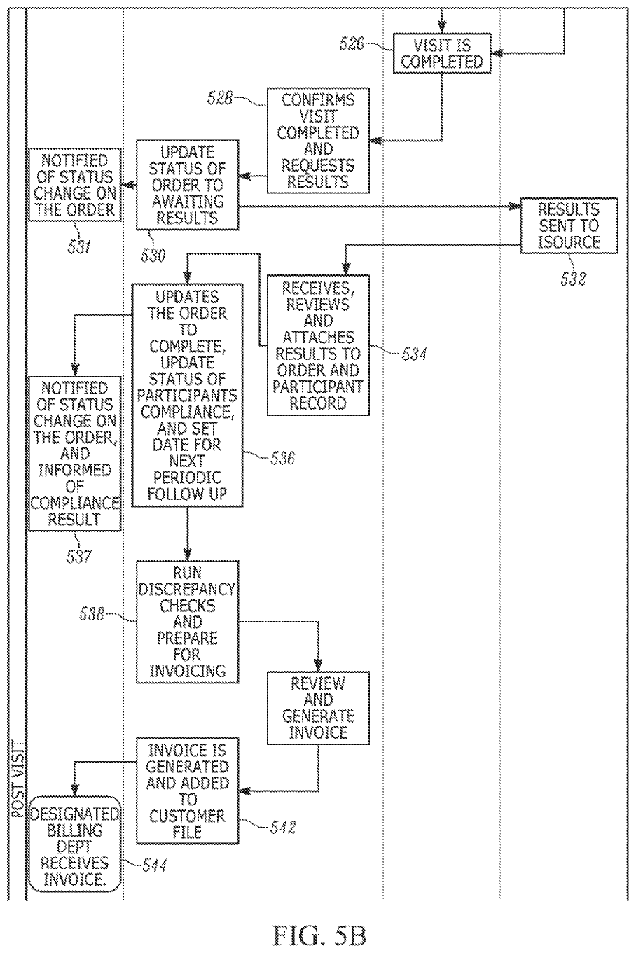

An alternative method 500 of using the occupational health system is illustrated in FIGS. 5A-5B. Throughout the method 500, several steps are performed by the system 100 (“SourceLink™”). When the system tasks are completed, a notification is automatically sent to the next user to act (the customer, 1 Source Team member, Participant, or Clinic). Each of these users have their own terminal or portal through which they can access the system. Through the participant portal, the injured employee of the customer can log in and securely review their medical/screening records. The method 500 involves less steps taken by a team member, which can reduce costs. 5A illustrates the pre-visit steps. In step 502, a customer creates a new order as discussed above. The order is entered into the system in step 504 and the status is set to “awaiting customer review”. The customer reviews the order in step 506, and in step 508 determines if it is correct. If any errors are found, they are corrected and the customer returns to step 506 to review the revised order. If the order is correct, the customer submits the order to the system in step 510. The system adds the new order to the queue in step 512 so that it can be reviewed by a team member. A team member picks the order from the queue in step 514 and reviews it for completeness in step 516. If the order is incomplete, it is returned to the customer for review, restarting at step 508. If the order is complete, the system assigns a specific team member, a fulfillment rep, to the order in step 518. The customer is notified of the status change and the assignment of a rep in step 519. The rep then schedules a visit in step 520 and enters the scheduled visit into the system in step 522. The participant is notified of the scheduled visit by the system in step 523. The system further generates any necessary paperwork for the visit. In step 524, the participant receives from the system visit confirmation and any necessary paperwork ahead of the scheduled visit. Also, before the visit, the clinic receives authorization forms from the system in step 525.

FIG. 5B illustrates the steps of the method 500 that occur after the visit. The visit is completed in step 526. The rep confirms that the visit was completed and requests results of the visit in step 528. In step 530, the rep further updates the status to reflect that the visit has been completed. The system notifies the customer of this change in step 531.

Sometime after the visit, the clinic sends the results of the visit to the rep in step 532. The rep reviews the results, and attaches them to the order and to the participant's records in step 534. In step 536, the status is updated to indicate that the results and if necessary, follow up visits are scheduled. The customer is notified of these changes and the results in step 537. The system automatically runs discrepancy checks, to ensure that the results received from the clinic match what was requested in the order, and then prepares an invoice for the visit in step 538. The rep reviews the invoice in step 540 and confirms that it is correct in the system in step 542. The system then sends the invoice to the customer, and the customer receives it at the designated billing department in step 544. The customer then pays the invoice and the order is complete.

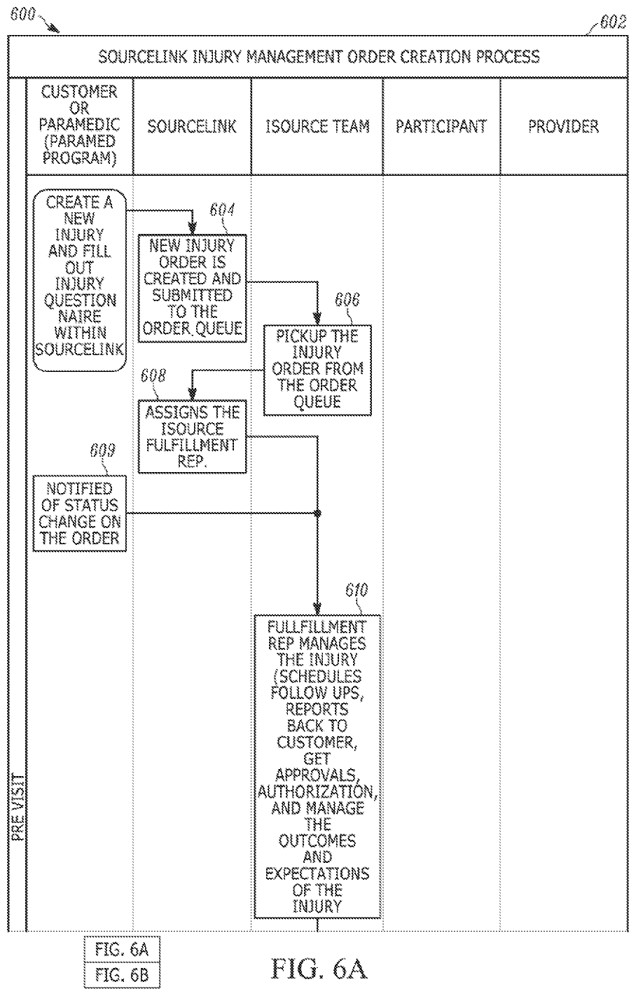

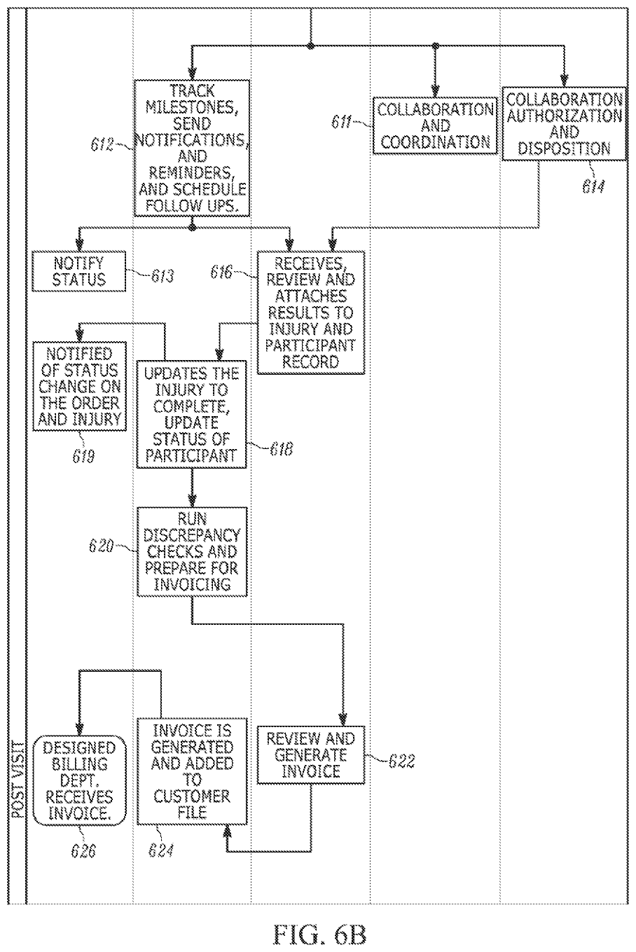

FIGS. 6A-B illustrate an exemplary method 600 of using an occupational therapy system in accordance with some embodiments contemplated herein. The method 600 illustrates using the management system to arrange and organize the treatment of an injury that requires ongoing treatment. The method 600 can be performed by any user through one of the customer portals or specifically by a paramedic using the Paramed app described above. The “provider” is the health care provider, such as a clinic or physical therapist. In steps 602-608, a new injury order is submitted by the customer, added to a queue by the system, reviewed by a team member, and assigned to a rep as described in the previous methods above. Once the injury is assigned, the customer is notified in step 609. In step 610, the fulfillment rep manages the injury by scheduling a series of appointments, follow ups, transmitting reports to the customer, getting approval from the customer and participant, submitting authorization forms to the clinic and handling the other necessary paperwork, and reviewing and managing results in step 610. Managing the injury involves tracking the milestones and sending notification of said milestones in step 612, which is done by the system. It further includes remaining in contact and collaborating with both the clinic and the participant in steps 611 and 614. The customer is periodically notified of changes in status in step 613. Records are also periodically received and reviewed by the rep in step 616. Once the full treatment process is completed, the system updates the status to complete in step 618 and notifies the participant. The system further updates the customer in step 619 of the completion. Once complete, the system runs a discrepancy check to ensure that the treatment that was completed matched the original injury form in step 620. If there are no discrepancies, an invoice is prepared in step 620 and sent to the rep for review in step 622. Once the invoice is approved by the rep, the system generates the invoice in step 624 and adds it to the customer file on the system. The designated billing department of the customer receives the invoice in step 626. In addition to generating, reviewing and distributing invoices to the billing department of the client, the system also generates, reviews, and distributes invoices to insurance providers to make and manage insurance claims.

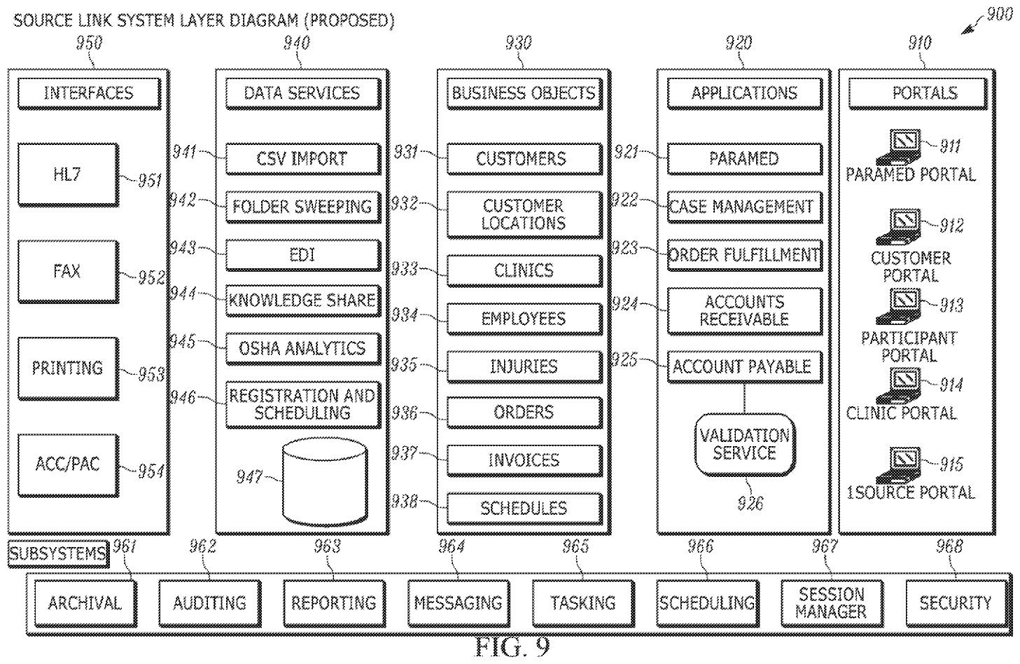

FIG. 9 illustrates a system layer diagram of the management system 900 described herein being used for injury management. The system 900 includes a series of portals 910. The portals 910 include the paramedic portal 911 described above, the customer portal 912 described above, a participant portal 913, clinic portal 914, and team portal 915 (shown as “1Source Portal”). The participant portal 913, is any computing device by which the participant, the employee of the customer seeking treatment, can access the system 900 to see the status of their orders, and their profile. The clinic portal 914 is any computing device through which clinics included in the network can access the network. Through the clinic portal, practitioners can access the guidelines of how to provide treatment to providers. They can further check histories of the participants, check scheduled appointments, review paperwork and authorization forms submitted by the team member, and transmit results to the system. The team portal 915 is any computer that a team member can use to access the system. Through team portals 915, team members can take any of the steps described in FIGS. 4-6, as well as review the files of customers, participants, and clinics in the system.

The system 900 operates a number of applications 920. These include applications for each of the modules described in FIG. 1 above. Specifically shown are applications within the injury management module. Additionally, FIG. 9 does not provide a comprehensive list of injury management applications, it is merely an exemplary list of applications to show the overall system layout. The paramedic application 921 is described above. A case management application 922 is usable to manage the individual customers or participants or individual injuries. The order fulfillment application 923 is usable to fulfill individual orders. This application facilitates communications on the order between the customer, team member, clinic, and participant. It further identifies the correct clinics and the correct guidelines based on the order submitted. Accounts receivable 924 and accounts payable 925 facilitate the invoicing of the customer and payment of the clinics. The validation service application 926 is usable to receive results from the clinic, analyze those results to determine if further treatment is needed, and compare those results to the order for discrepancies as described above.

The system 900 further includes several business objects 930. The business objects 930 include a plurality of customers 931, each customer having one or more locations 932, the network of clinics 933, the employees of the customers (or participants) 934. The business objects 930 also includes any injuries 935, orders 936, invoices 937, and scheduled visits 938 being managed by the system.

Managing all of the above mentioned applications 920 and business objects 930 requires handling a great deal of data. As a result, the system 900 includes multiple data services 940. Comma separated values import 941, folder sweeping 942, electronic data interchange 943, and knowledge share 944, and registration and scheduling 946 are all commonly used data services. OSHA analytics 945 is an industry specific set of analytics used to report mass amounts of data regarding jobsite injuries to OSHA, which they then use to improve regulation. Finally, the data services 940 include the storing of the data 947. The acquired data can be used for a number of tasks outside of OSHA analytics. For example, the data services automatically track reported injuries, damage, and near misses in order to detect trends. These trends can be used to more accurately and quickly identify risks so that the customer can address the identified risk to prevent future damage and injury. Data from a plurality of customers can be used to identify and analyze industry wide trends for even more accurate risk management. In other modules, the data services 940 are used to process employment data, advertising/marketing data, project expenses and times, client or partner relationships, etc. Having data from all of these different aspects of management stored within a single system enables a great deal of data analytics to help optimize management.

The system 900 includes a number of interfaces 950 to interact with people or outside systems. These interfaces include fax 952 and printing 953 for creating physical copies of records. The interfaces further include a means to interface with accounting software, such as ACC/PAC 954, and a means of exchanging health information through HIPAA approved means such as HL7 951.

A series of subsystems 960 makeup the system 900. Archival 961 is used to backup and maintain older data in a more efficient manner. This includes compressing the data to take up less memory. The auditing subsystem 962 keeps data on system use so that a user can determine what applications, business objects, or other subsystems 960 are using a disproportionate amount of resources. This can be used to detect fraud, or some other security leak. It can further be used to better optimize the system 900 to reduce waste. Auditing further monitors who makes certain changes to data 947 and the system 900 for future reference. The reporting subsystem 963 notifies listeners of certain events such as a status change or appointment reminder. A messaging subsystem 964 facilitates communication between users of the system, such as between the team member and the customers, clinics, and participants. The tasking subsystem 965 maintains the queue of orders and assigns those orders to specific team members or reps. The scheduling subsystem 966 maintains a list of all scheduled visits or other scheduled events, the parties participating, and other listeners that should be notified of the event. The session manager 967 becomes active when a user accesses the system 900 and monitors the user's usage of the system. If the user has become inactive for a predetermined period of time, the session manager 967 logs the user off the system to weed out any non-active users and protect the user in case they forgot to log off themselves (particularly if on a non-secure terminal). Finally, a security subsystem is used to protect data on the system 900 and communication over the system 900 from being accessed by unauthorized persons.

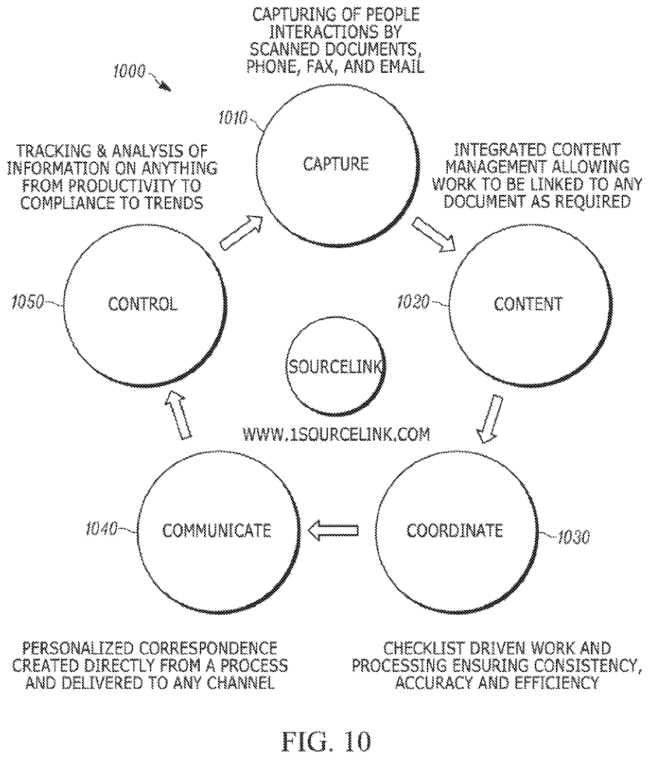

FIG. 10 illustrates a cycle diagram 1000 for exemplary systems, methods and apparatus in accordance with some embodiments of the invention. The cycle diagram 1000 shows features of the system described above. The first feature, capture 1010, refers to the saving of documents and communications made in the fulfillment of the orders described above. This includes the initial order, communications between the team member, customer, participant, and clinic, results sent from the clinic, forms submitted to the clinic, forms and records created for insurance or regulatory agencies, etc. As described above, several of these documents are created within the system. Those that are not, are scanned into the system. This way, a complete record of the treatment is in a single location if it needs to be found at a later date. This saves time and resources that would otherwise be used searching the archives of multiple entities and multiple communications methods in order to compile all of the same documents.

With all of the documents captured 1010 in a single location, the system can control the content 1020. This feature involves linking all related content together so that it can easily be navigated. As described above, the records can be found by navigating through the participant, the employer, or the clinic. It can be further divided by type of treatment, location, time, or any other of a number of criteria. Through this content management, large amounts of data can be easily analyzed to detect trends or search for waste. Additionally, complete records of a single customer, location, client, clinic, or injury type can be grouped together if needed. This can once again reduce the amount of time needed to comply when a clinic needs a participants full history, or a regulatory agency or insurance agency requires full records of a certain injury type.

The content management 1020 additionally serves to facilitate coordination 1030. The system generates checklists for a rep to follow when managing an order or case. With every document added to the system, and all of the content tied together, it is easy to go down the checklist and ensure that each step is filled. Many of the steps can be automatically taken in response to certain documents being received in the system as described above. This level of coordination 1030 expedites the treatment process and reduces the number of errors and oversights.

The system also facilitates communication 1040 between the various users (customers, team members/reps, participants, and clinics). As stated above, several types of communication 1040 are automatically generated and sent as a result of certain precursor steps being fulfilled. Communication through the system further provides a convenient backlog so that entire histories can be searched.

Each of the other features combine to allow for control 1050. Control 1050 broadly includes any tracking and/or analysis of anything on the system. As stated above, this can include tracking and analyzing trends of certain customer, locations, participants, injury types, or clinics. Control 1050 over the data can be used to detect treatment effectiveness so that guidelines can be amended, or to track price trends. It can further be used to comply with government oversight and to detect waste and/or fraud.

FIG. 11 illustrates a flow chart illustrating an exemplary method 1100 of visiting a clinic. First the new clinic must register with the system 1102. Once registered, authorization forms are verified 1104. As described above, clinics are used both for regular visits to comply with regulation 1110 as well as visits to treat injures 1150. There are two types of regulatory visits, fast track visits 1120 and screening exams 1130. In both types of visits the authorization form is compared to a flow sheet illustrating the tasks required by the regulation 1121/1131. If the tasks are authorized, the tasks are then performed 1122/1132. Once the fast track tasks are performed, all of the forms are double checked 1123. If the forms are correct, a confirmation is place in the chart 1125. In cases where the task performed was a drug test or blood alcohol test (or similar task) the chain of custody paperwork is completed and the samples are properly sealed 1124. When the records are received, receipt is confirmed 1190.

After the tasks are performed for a screening exam visit, the results are organized into a chart 1233. The patient is then placed in a visiting room to be seen by a doctor 1134. If additional assistance is required, the clinic staff provide the assistance to the patient 1180. If not, the patient checks out and if needed schedules a follow up appointment 1182. When the system 100 receives records of the visit, receipt is confirmed 1190.

In an injury visit, the authorization is compared to a flow sheet of required tasks to ensure all tasks are authorized 1152. The clinic staff determines if the circumstances of the injury and/or behavior of the patient necessitate a drug screen and/or blood alcohol test 1154. If so, said tests are performed 1156. Regardless of whether or not a drug screen and/or blood alcohol test are administered, triage is performed 1158 to determine the extent of the injury and what treatment is needed. The information gathered is recorded in intake forms 1160 and the patient is placed in a visiting room 1162. A medical provider evaluates the patient 1164 and determines whether or not a medical procedure is required 1166. If so, the patient is assisted as required 1180. Once all necessary assistance is provided, the patient checks out 1182 as described above.

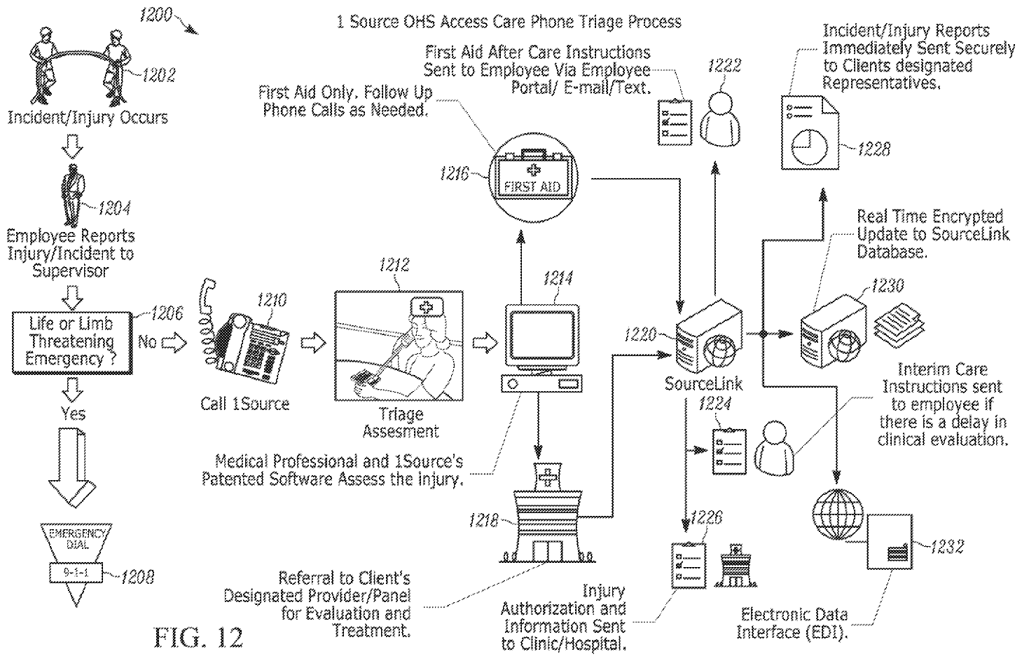

FIG. 12 illustrates an example scenario 1200 of injury management under the system 100. In the scenario 1200, an employee is injured on the job 1202. The employee reports the injury to their immediate supervisor 1204. First the employee and/or supervisor determine if the injury is an emergency 1206. If so, 9-1-1 is dialed 1208. If it is not an emergency, the system 100 is called 1210. The system 1210 connects the user to a medical professional to provide phone triage 1212 as described above. The medical professional that performs the phone triage determines if first aid and/or a clinic visit are required and refers the user accordingly 1214. If only first aid is required, the first aid is provided as described above 1216. First aid only does not require a workers compensation claim. By determining whether or not first aid is sufficient via phone triage, the system 100 reduces the number of unnecessary workers compensation claims. The paramedic enters the information from the encounter into the system 1220.

If the medical professional determines that additional medical treatment is needed, the user is referred to a clinic 1218 as described above. When the visit is made, the clinic referral is entered into the system 1220. The system 100 sends after care instructions to the client after first aid via the user portal, email, or text message 1222. The aftercare instructions include activities like home exercises or stretches to help the injury heal without requiring clinic referrals in order to reduce workers compensation claims.

If a clinic visit is required, the system sends the user interim care instructions to help manage the injury until a visit can be arranged 1124. The system additionally sends the injury authorization and the event information to a clinic or hospital to schedule an appointment 1126.

The system 100 transmits and/or makes the incident report available over a number of secure means. The injury report is immediately and automatically sent to a predesignated representative of the client 1228. The report is also encrypted and stored in the system database such that it can be accessed by the client 1230. The system also provides an electronic data interface to allow the data to be shared between authorized parties 1232.

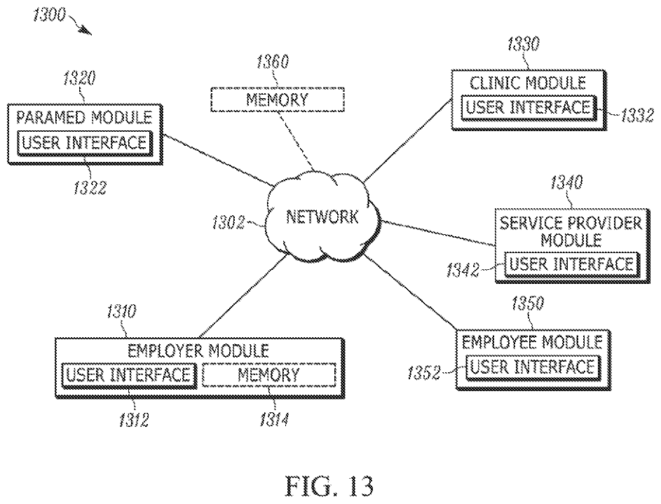

FIG. 13 is a network diagram on which the system 100 operates. The network includes a number of terminals or modules 1310-1350 communicatively coupled via a network 1302. An employer module 1310 is a computer used by a designated representative of a client, such as a human resources manager, to access the system. The module 1310 includes a user interface, such as a touchscreen or standard monitor, keyboard, and mouse set up with which the user can submit and receive information from the system. In some embodiments, the employer module 1310 stores all of the relevant data locally in memory 1314 so that it can be accessed without an internet access. In alternative embodiments, the data is stored remotely in server memory 1360.

The paramed module 1320 is a portable computer used by a paramedic to operate the paramed app described above. As discussed, the module 1320 can be a standard smart phone or tablet having a touchscreen user interface 1322. The clinic module 1330 is a computer device at a network clinic used to access the network. It includes a user interface 1332 through which clinic personnel can access the network to submit and receive information. The service provider module 1340 is a computer operated by system administrators to perform the tasks described above. They perform these tasks through a user interface 1342. In some forms, individual employees have modules 1350 they can use to access the network through employee portals. These modules 1350 can be personal or work computers and/or portable devices such as smartphones or tablets. The employee modules 1350 include user interfaces 1352.

As discussed above, the network enables secure, direct communication between the users of the system. Certain information is automatically shared between users and/or is stored in memory 1360 where it can be access by authorized users.

A number of examples of the system and related methods described herein are provided below. These are intended to illustrate some examples, not the invention as a whole.

In one form a management system is provided comprising a master interface providing access to a regulatory compliance module, an injury management module, a client relations management module, an employee health and safety module, a human relations module, and a project management module.

In some forms of the system above, the modules are configured to be controlled by a third party in order to manage some aspects of the client a company.

In alternative forms of the exemplary system, the modules are controlled by an employee of to manage a company employing the employee.

In another exemplary form, an apparatus comprising at least one main computer, the main computer being one of a group consisting of a server, a smart phone, a tablet, a laptop, and a desktop computer is provided. At least one customer portal is in communication with the at least one main computer, the customer portal being one of a group consisting of a smart phone, a tablet, a laptop, and a desktop computer. At least one provider portal is also in communication with the at least one main computer, the provider portal being one of a group consisting of a smart phone, a tablet, a laptop, and a desktop computer. The main computer has a storage medium storing a computer program executable to execute steps comprising:

receiving an order from the customer portal, the order requesting treatment for a participant;

receiving availability data from the participant; selecting a treatment provider from a network of providers based on the treatment requested treatment;

transmitting a request for an appointment to the provider portal; and transmitting guidelines to the provider portal, the guidelines describing how to provide the requested treatment.

In addition to the apparatuses and system described herein, a number of methods are also considered. One such method is a method of providing occupational health services comprising:

receiving an order from a company, the order requesting treatment for an employee of the company;

receiving availability from the employee; selecting a treatment provider from a network of providers based on the treatment requested treatment;

scheduling an appointment with the provider based on the availability received from the employee; and providing guidelines to the provider on how to provide the requested treatment.

In some embodiments, this exemplary method further includes:

receiving results from the provider;

comparing the results to the guidelines; and

determining if further treatment is necessary.

In alternative embodiments, this exemplary method further includes:

receiving an invoice from the provider;

comparing the invoice to the order requesting treatment and the guidelines; and

transmitting the invoice to the company.

In one form, the system described above is a system for managing occupation services comprising:

at least one customer, the customer being a business having a plurality of employees;

a network of treatment providers;

a list having services provided by each treatment provider in the network, the location of each provider in the network, and the operating hours of each provider in the network;

a portal operable by the customer to request treatment for one of the plurality of employees;

an application operable to compare the requested treatment to the list and returning a clinic that provides the required services, and is located near the employee and is operating when the employee is available; and

a list of guidelines describing how to treat injuries, the list being accessible by the providers in the network.

The example system may further comprise a paramedic portal through the list of guidelines can be accessed by a paramedic.

Also considered herein is a non-transitory storage medium storing a computer program executable by a processor based system, the computer program causing the processor based system to execute steps comprising:

receiving an order from a company, the order requesting treatment for an employee of the company;

receiving availability from the employee;

selecting a treatment provider from a network of providers based on the treatment requested treatment;

scheduling an appointment with the provider based on the availability received from the employee; and

providing guidelines to the provider on how to provide the requested treatment.

A second exemplary method is a method of providing first aid comprising:

providing a plurality of questions to a paramedic via a portable computing device;

receiving answers to the questions from the paramedic;

providing additional questions based on the answers received;

receiving additional answers to the additional questions;

displaying instructions for first aid procedure based on additional answers.

The second exemplary method may further comprise:

displaying a paper doll on the portable computing device;

receiving information indicating selection of a body part on the paper doll; and

providing the plurality of questions based on the selected body part.

In addition to the above-mentioned embodiments, it should be understood that a variety of methods are also disclosed herein. For example, methods of setting up a system are disclosed herein, as are methods for sharing information such as guidelines and scheduling and for use in providing occupational therapy. These and other methods related to the subject matter set forth herein are intended to be covered by this disclosure. It should also be understood that while certain features have been described with certain embodiments, these features may be intermixed or interchanged with one another to form other embodiments as desired. All features disclosed herein are intended to be used in any of the embodiments disclosed herein either in lieu of similar features or in combination with other features.

This detailed description refers to specific examples in the drawings and illustrations. These examples are described in sufficient detail to enable those skilled in the art to practice the inventive subject matter. These examples also serve to illustrate how the inventive subject matter can be applied to various purposes or embodiments. Other embodiments are included within the inventive subject matter, as logical, mechanical, electrical, and other changes can be made to the example embodiments described herein. Features of various embodiments described herein, however essential to the example embodiments in which they are incorporated, do not limit the inventive subject matter as a whole, and any reference to the invention, its elements, operation, and application are not limiting as a whole, but serve only to define these example embodiments. This detailed description does not, therefore, limit embodiments of the invention, which are defined only by the appended claims. Each of the embodiments described herein are contemplated as falling within the inventive subject matter, which is set forth in the following claims.

What is claimed is:

1. A system for providing regulatory compliance services comprising:
a processor-based system having an interface giving access to a service to aid an employer in complying with regulations, the processor-based system configured to:
receive, via the interface, job specific information pertaining to an employee position from the employer at a first computing device;
identify one or more regulatory requirements for the employee position based at least in part on the received job specific information;
create, for the employee position, a program for compliance with the identified one or more regulatory requirements;
automatically schedule a screening appointment for a worker associated with the employment position based at least in part on the program for compliance; and
notify the worker, via the system, of the scheduled screening appointment.

2. The system of claim 1 wherein the processor-based system is further configured to:
receive, via the interface, a screening result from a clinic or medical review officer at a second computing device.

3. The system of claim 2 wherein the processor-based system is further configured to:
provide the worker with a first form for the scheduled screening appointment;
provide a clinic or medical review officer with a second form for the scheduled appointment;
generate an invoice or bill for the scheduled appointment; and
transmit the invoice or bill to an employer.

4. The system of claim 1 wherein the screening appointment includes at least one of a pre-employment fitness screening, random testing, testing as a result of probable cause, post-accident or incident testing, follow up testing, and periodic worker fitness screening.

5. The system of claim 1 wherein identifying one or more regulatory requirements includes searching a database including regulations of at least one of the government, parent company, industry groups, international bodies, ratings organizations, certifying organizations, and consumers.

6. The system of claim 5 wherein identifying one or more regulatory requirements includes compiling a list of regulatory requirements applicable to the job specific information.

7. The system of claim 1 wherein receiving the job specific information includes receiving information pertaining to at least one of a physical demand, job demand, and job task, location, and equipment operated.

8. The system of claim 1 wherein the worker is at least one of employed in the employee position and a candidate for the employee position.

9. The system of claim 1 wherein the program for compliance includes one or more tasks to be completed by the worker pre-employment and one or more tasks to be completed during employment to maintain compliant with the one or more regulations.

10. The system of claim 1 wherein the one or more regulatory requirements for the employee position include federal, state, and industry regulations issued by federal, state, and industry regulatory bodies and the processor-based system is configured to identify those federal, state and industry regulations; and
the program for compliance created by the processor based system is crafted from those federal, state and industry regulations, and addresses occupational health concerns for that employee position in an effort to reduce the likelihood of the worker being injured while performing in the employee position or, if injured while performing duties related to the employee position, increase efficiency of the worker's recovery efforts so that the worker can return to the employee position more quickly.

11. The system of claim 10 wherein, if the worker is injured while performing duties in the employee position, the processor-based system creates for the worker and medical personnel assisting the worker in occupational therapy a treatment plan to be followed to increase the efficiency of the worker's rehabilitation efforts and return the worker to the employee position more quickly.
